(19) United States
(12) Patent Application Publication
(10) Pub. No.: US 2022/0298547 A1
(43) Pub. Date: Sep. 22, 2022

(54) Multiplex Viral Pathogen Analysis and Uses Thereof

(71) Applicant: Michael E. Hogan, Tucson, AZ (US)

(72) Inventor: Michael E. Hogan, Tucson, AZ (US)

(73) Assignee: PathogenDx, Inc., Scottsdale, AZ (US)

(21) Appl. No.: 17/698,400

(22) Filed: Mar. 18, 2022

Related U.S. Application Data
(60) Provisional application No. 63/163,423, filed on Mar. 19, 2021.

(51) Int. Cl. C12Q 1/686; C12Q 1/70; C12Q 1/689; C12Q 1/6853; C12Q 1/6874

(52) U.S. Cl. CPC C12Q 1/686; C12Q 1/701; C12Q 1/689; C12Q 1/6853; C12Q 1/6874

(57) ABSTRACT

Provided herein are methods for isolating, detecting and analyzing a virus in a sample. Generally, the virus is isolated via centrifuging, incubating, aggregating, and lysing the sample to obtain a crude neutralized lysate. The crude neutralized lysate is analyzed and the virus(es) are detected by performing one of a first polymerase chain reaction (PCR) amplification followed by a second PCR amplification, by an isothermal amplification or by the reverse transcription reaction and the first polymerase chain reaction amplification together utilizing fluorescently labeled primer pairs and hybridizing the resultant fluorescent labeled amplicons to complementary nucleic acid probes.

### CROSS-REFERENCE TO RELATED APPLICATIONS

This non-provisional patent application claims benefit of priority under 35 U.S.C. § 119(e) of provisional application U.S. Ser. No. 63/163,423, filed Mar. 19, 2021, the entirety of which is hereby incorporated by reference.

### BACKGROUND OF THE INVENTION

### Field of the Invention

The present invention relates to the field of RNA based pathogen analysis. More particularly, the present invention relates to viral pathogen isolation and analysis in plant, livestock and human samples or environmental samples using a multiplex assay.

### Description of the Related Art

Single stranded RNA viruses are well known causative agents in human and animal disease (HIV, hepatitis C, measles, influenza, coronavirus and other respiratory viruses) and plant disease (tobacco mosaic virus, cucumber mosaic virus, Potyvirus) and many others. Those viruses each comprise a single stranded RNA genome in the 5 kb-40 kb range and a small number of coat or nucleic acid binding proteins which form the overall structure of the virion. Single stranded RNA viruses typically have a rod-like or a spherical icosahedral shape with an outer dimension <1 μm.

The prior art is deficient in methods of detecting the presence of disease causing virus contamination in plants, animals, human sources, air, water, swabs and other surface collection methods. Particularly, the prior art is deficient in methods of isolating viruses from a sample such that the viral nucleic acids can be processed from the collected sample without explicit extraction. The present invention fulfills this long-standing need and desire in the art.

### SUMMARY OF THE INVENTION

The present invention is directed to a method for isolating at least one virus in a sample for analysis. In the method, the sample is obtained and is fluidized to produce a suspension comprising the virus. The suspension is centrifuged to obtain a first supernatant comprising the virus and the first supernatant is centrifuged to obtain a second supernatant comprising the virus. The second supernatant is incubated with a water soluble high molecular weight polymer or non-viral particles or a combination thereof and the pH is adjusted to less than pH 6 to form an aggregated virus. The aggregated virus is centrifuged to obtain a pellet comprising the aggregated virus. A lysis buffer is added to the pellet, the pellet is heated in the lysis buffer and neutralized to pH to 7 to produce a crude neutralized lysate comprising at least one viral ribonucleic acid.

The present invention is directed to a related method further comprising analyzing the crude neutralized lysate to detect the presence of the virus in the sample. In the related method viral nucleic acids are isolated from the crude neutralized lysate. A reverse transcription reaction is performed using the isolated viral nucleic acids as template to obtain virus-specific complementary deoxyribonucleic acids (cDNAs). In at least one amplification, a target nucleotide sequence is amplified in the virus-specific cDNA using at least one pair of primers selective for the virus to generate a plurality of virus-specific amplicons. The presence of the at least one virus in the sample via hybridization of the plurality of virus-specific amplicons to a plurality of nucleic acid probes each specific for the virus.

The present invention is directed to another related method of analyzing the crude neutralized lysate. In the method, each of the primer pairs further comprises a first fluorescent label. A plurality of first fluorescent labeled virus-specific amplicons are generated thereby.

These and other features, aspects, and advantages of the embodiments of the present disclosure will become better understood when the following detailed description is read with reference to the accompanying drawings.

### BRIEF DESCRIPTION OF THE DRAWINGS

FIG. 1 is a flow chart showing the steps to isolate and detect RNA virus in a sample.

### DETAILED DESCRIPTION OF THE INVENTION

The articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

The term “or” in the claims refers to “and/or” unless explicitly indicated to refer to alternatives only or the alternatives are mutually exclusive, although the disclosure supports a definition that refers to only alternatives and “and/or”.

The terms “comprise” and “comprising” are used in the inclusive, open sense, meaning that additional elements may be included.

The term “including” is used herein to mean “including, but not limited to”. “Including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure. For example, a stated pH of about 7 to about 10 encompasses a pH of 6.3 to 11.

In one embodiment of this invention, there is provided a method for isolating at least one virus from a sample, comprising the steps of obtaining the sample; fluidizing the sample to produce a suspension comprising the virus; centrifuging the suspension to obtain a first supernatant comprising the virus; centrifuging the first supernatant to obtain a second supernatant comprising the virus; incubating the second supernatant with a water soluble high molecular weight polymer or non-viral particles or a combination thereof; adjusting the pH to less than pH 6 to form an aggregated virus; centrifuging the aggregated virus to obtain a pellet comprising the aggregated virus; adding a lysis buffer to the pellet; heating the pellet in the lysis buffer; and neutralizing the pH to 7 to produce a crude neutralized lysate comprising the one virus.

In this embodiment, the sample may be a tissue or cells from a human, a plant, an animal, bacteria, a fungus or algae, or a combination of these. Particularly, the sample may comprise surface swabs, skin swabs, a nares (nostril) swab, milk, blood, sputum, mucus, urine, or feces. Alternatively, the sample may be an aerosol.

In one aspect, the sample may be harvested from an aerosol, which is dispersed into a liquid. In a second aspect, the sample may be harvested by washing with a liquid. In a third aspect, the sample may be harvested by dispersion into a liquid. In a fourth aspect, the sample may be harvested by dispersing a swab taken off a surface into a liquid. In all aspects, the liquid is water or a buffer. In all aspects, when the volume is large, the sample may be concentrated by filtration.

In this embodiment, the virus may be a single stranded RNA virus or a double stranded RNA virus. Examples of such viruses include, but are not limited to, a human immunodeficiency virus, an influenza virus, a SARS virus, a coronavirus, a COVID-19, a hepatitis C virus, a dengue virus, a norovirus, a rotavirus, a measles virus, a tobacco mosaic virus, a tobacco ringspot virus, a hemp mosaic virus, a cucumber mosaic virus, a potyvirus, a beet curly top virus, a tobacco streak virus, an alfalfa mosaic virus, an arabis mosaic virus, a cannabis cryptic virus, a hop latent virus, a hemp streak virus or a cauliflower mosaic virus. A combination of these viruses may also be detected simultaneously.

In this embodiment, the method may comprise isolating a crude viral nucleic acid preparation comprising at least one virus, such as an RNA virus. The nucleic acids may be obtained by fluidizing the sample to obtain a suspension. Fluidization may be performed by any mechanical means including, but not limited to, a pestle, a sonicator or a Parr bomb. Fluidizing may be performed in buffers, for example, but not limited to, a disruption buffer having a pH from about 7 to about 8. Also, the buffers may comprise detergents including, but not limited to, Tween-20, Triton-X100, NP-40 and CHAPS. The fluidized sample may be centrifuged at a temperature of about 15° C. to about 30° C., and at a speed from about 800×g to about 1,000×g. Centrifugation results in a pellet comprising cell debris and other debris, such as non-dissolved solids, and a first supernatant comprising intact virus, bacterial cells, algae cells, and fungal cells. In addition, the first supernatant may be centrifuged at a temperature of about 15° C. to about 30° C., and at a speed of about 10,000 to about 15,000×g. Centrifugation results in a pellet comprising bacterial cells, algae cells and fungal cells and a second supernatant comprising the intact virus.

In this embodiment, a water soluble high molecular weight polymer may be added to the second supernatant and the pH may be adjusted from about pH 3 to about pH 5 to aggregate the virus. The water soluble high molecular weight polymer may be at a concentration of 1% to 10% w/v. The step of incubating may be performed at a pH from about 3 to about 5. Furthermore, the step of incubating may be performed at a temperature between about 15° C. and about 30° C. Further still the step of incubating may be performed for about 10 min to about 30 min. Further still the heating step may be performed at a temperature of about 60° C. to about 80° C. Further still, the step of harvesting the sample may comprises washing the sample or dispersing a swab in a liquid.

Aggregation of the virus may be caused by neutralization of its surface charge to a value near zero. Examples of water soluble high molecular weight polymers include, but are not limited to, a polyethylene glycol, a dextran or a dextran sulfate, or a combination of these. In this embodiment, the pH may be adjusted by the addition of a weak acid including, but not limited to, a sodium acetate or an acetic acid.

Virus aggregation may be facilitated by the addition of non-viral particles. Non-viral particles include, but are not limited to, a ceramic particle. Silicon dioxide (SiO₂) is an example of a ceramic particle. In one aspect of this embodiment, a combination of viral and non-viral particles may be used. The non-viral particle may have an overall dimension from about 50 nm to about 500 nm. Furthermore, the second supernatant comprising the aggregated virus may be centrifuged at a speed from about 10,000×g to about 15,000×g to obtain a pellet of aggregated virus.

In this embodiment, the nucleic acids in the aggregated virus may be released by addition of a lysis buffer to the pellet, followed by heating at a temperature between about 60° and about 80° for about 10 min. Any lysis buffer having a pKa from about pH 7 to about 10 may be used for this purpose. For example, the lysis buffer may be 10 mM Tris buffer pH 8. After heat incubation, the lysed virus sample may be neutralized to pH of about 7 to obtain a crude neutralized lysate comprising at least one viral ribonucleic acid. The crude lysate, which comprises nucleic acids including viral ribonucleic acids, may be used in the subsequent steps without further purification.

Further to this embodiment, the method comprises analyzing the crude neutralized lysate to detect the presence of the virus in the sample where the method comprises the steps of isolating, from the crude neutralized lysate, viral nucleic acids; performing a reverse transcription reaction using the isolated viral nucleic acids as a template to obtain virus-specific complementary deoxyribonucleic acids (cDNA); amplifying, in at least one amplification, a target nucleotide sequence in the virus-specific cDNA using at least one primer pair selective for the virus to generate a plurality of virus-specific amplicons; and detecting the presence of the at least one virus in the sample via hybridization of the plurality of virus-specific amplicons to a plurality of nucleic acid probes each specific for the virus. Commercially available reverse transcriptase enzyme and buffers are used for this purpose. Amplification may be performed using any method including, but not limited to a single PCR reaction, a tandem PCR reaction, qPCR, real-time PCR, PCR-CE, PCR-Sanger sequencing, PCR-Next Generation Sequencing, a gel based PCR and loop-mediated isothermal amplification (LAMP). In one aspect, amplification is by PCR.

In another further embodiment each of the primer pairs comprises a first fluorescent label, where the method generates a plurality of first fluorescent labeled virus-specific amplicons. In one aspect, a single amplification may be performed wherein the primers are fluorescently labeled to generate a plurality of fluorescent labeled amplicons. In another aspect, the amplification may be by tandem PCR that comprises two amplification steps wherein the first amplification uses non-fluorescent primers to generate a plurality of non-fluorescent amplicons. The non-fluorescent amplicons are used as template for a second amplification that uses fluorescent primers having a sequence complementary to an internal flanking region in the virus-specific amplicons to generate a plurality of fluorescent amplicons. In both aspects of the quantitating the plurality of amplicons generated detect presence of the virus in the sample.

Alternatively, the fluorescent labeled amplicons may be hybridized to a plurality of nucleic acid probes directly covalently linked to a solid support. Each nucleic acid probe has a sequence specific to a virus among the plurality of viruses. In this embodiment, the solid support may be any solid microarray support including, but not limited to, a 3-dimensional lattice microarray. The solid microarray support may be microarray is made of any suitable material known in the art including, but not limited to, borosilicate glass, a thermoplastic acrylic resin, for example, poly(methylmethacrylate-VSUVT) a cycloolefin polymer, for example, ZEONOR® 1060R), a metal including, but not limited to, gold and platinum, a plastic including, but not limited to, polyethylene terephthalate, polycarbonate, nylon, a ceramic including, but not limited to, titanium dioxide (TiO₂), and indium tin oxide (ITO) and engineered carbon surfaces including, but not limited to, graphene. A combination of these materials may also be used.

The solid support has a front surface and a back surface and is activated on the front surface by chemically activatable groups for attachment of the nucleic acid probes or linkers to which the nucleic acid probes are attached. The chemically activatable groups include, but are not limited to, epoxysilane, isocyanate, succinimide, carbodiimide, aldehyde, or maleimide. These are well known in the art and one of ordinary skill in this art would be able to readily functionalize any of these supports as desired. In a preferred embodiment, the solid support is epoxysilane functionalized borosilicate glass support.

Alternatively, the nucleic acid probes may be indirectly attached to the support using bifunctional polymer linkers. The plurality of bifunctional polymer linkers may be covalently attached to the chemically activatable groups in the solid support by a first reactive moiety at one end of the linker and covalently attached to one of the nucleic acid probes by a second reactive moiety at another position along. Examples of first reactive moieties include, but are not limited to, an amine group, a thiol group and an aldehyde group. In one aspect the first reactive moiety is an amine group. In one aspect, the first reactive moiety is an amine group. Examples of second reactive moieties include but are not limited to nucleotide bases like thymidine, adenine, guanine, cytidine, uracil and bromodeoxyuridine and amino acid like cysteine, phenylalanine, tyrosine glycine, serine, tryptophan, cystine, methionine, histidine, arginine and lysine. The bifunctional polymer linker may be an oligonucleotide such as OligodT, an amino polysaccharide such as chitosan, a polyamine such as spermine, spermidine, cadaverine and putrescine, a polyamino acid, with a lysine or histidine, or any other polymeric compounds with dual functional groups which can be attached to the chemically activatable solid support on the bottom end, and the nucleic acid probes on the top domain. Preferably, the bifunctional polymer linker is OligodT having an amine group at the 5′ end.

In another aspect of these further embodiments, the amplifying step and the detecting step may comprise performing one of a first polymerase chain reaction (PCR) amplification followed by a second PCR amplification, an isothermal amplification or the reverse transcription reaction and the first polymerase chain reaction amplification together, each of said amplifications using the at least one virus-specific complementary deoxyribonucleic acid as template and at least one first fluorescent labeled primer pair selective for the virus to generate the first fluorescent labeled virus-specific amplicons; hybridizing the first fluorescent labeled virus specific amplicons to a microarray comprising a plurality of nucleic acid probes each having a sequence corresponding to sequence determinants in a plurality of virus-specific ribonucleic acids; washing the microarray at least once; and imaging the microarray to detect a first fluorescent signal image corresponding to the first fluorescent labeled virus-specific amplicons.

In another aspect of these further embodiments, the amplifying step may comprise performing the first polymerase chain reaction using the at least one virus-specific complementary deoxyribonucleic acid as template and at least one unlabeled primer pair selective for the virus to generate virus-specific amplicons corresponding to the viral ribonucleic acid; and performing the second polymerase chain reaction using the virus-specific amplicons as template and the at least one first fluorescent labeled primer pair having a sequence complementary to a region in the virus-specific amplicons to generate the first fluorescent labeled virus specific amplicons.

In yet another aspect of these further embodiments, the microarray may comprise a plurality of bifunctional polymer linkers each covalently attached to the microarray and to one of the nucleic acid probes and each comprising a second fluorescent label covalently attached thereto, where the method further comprises detecting in the imaging step, a second fluorescent signal image corresponding to the second fluorescent labeled bifunctional polymer linkers; superimposing the first fluorescent signal image with the second fluorescent signal image to obtain a superimposed signal image; and comparing the sequence of the nucleic acid probe at one or more superimposed signal positions on the microarray with a database of signature sequence determinants for a plurality of viral ribonucleic acid thereby identifying the virus in the sample.

The second fluorescent label may be attached covalently to one or the other ends to the bifunctional polymer linker or may be attached covalently internally. The second fluorescent labels may be attached to any reactive group including, but not limited to, amine, thiol, aldehyde, sugar amido and carboxy on the bifunctional polymer linker. In one aspect, the bifunctional polymer linker is a CY5-labeled OligodT having an amino group attached at its 3′ terminus for covalent attachment to the activated surface on the solid microarray support. In this further aspect the second fluorescent label in the fluorescent labeled bifunctional polymer linker is different from the first fluorescent label in the first fluorescent labeled primer pair. The first fluorescent label and the second fluorescent label are a fluorescent dye selected from the group consisting of CY5, DYLIGHT™ DY647, ALEXA FLUOR 647, CY3, DYLIGHT DY547, and ALEXA FLUOR 550.

Having a fluorescent label (fluorescent tag) attached to the bifunctional polymer linker is beneficial since it enables the user to image and to detect the position of the individual nucleic acid probes (“spot”) printed on the microarray. By using two different fluorescent labels, a first fluorescent label for the amplicons generated from the crude neutralized lysate being queried and a second fluorescent label for the bifunctional polymer linker, the user may obtain a superimposed image that enables parallel detection of those nucleic acid probes which have been hybridized with amplicons. This is advantageous since it helps in identifying the virus(es) in the sample using suitable computer and software, assisted by a database correlating nucleic acid probe sequence and microarray location of this sequence with a known RNA signature in the viruses.

Provided herein are methods to isolate and analyze RNA viruses without the requirement for RNA extraction, via a simple process comprising a streamlined sample preparation combined with a highly parallel nucleic acid testing. The streamlined sample preparation is implemented within less than 2 hours without the need for refrigeration, using no specialized equipment other than an ordinary bench top centrifuge and a standard PCR thermal cycler. This aspect of the present invention constitutes a major simplification of the process by which a crude neutralized lysate is prepared from sample for analysis, at up to 100 samples in parallel and, in a practical sense, increases the speed and throughput in a way that enables very large (national-scale) testing of specimens, such as, diseased plant washes or human skin or nasal swab samples or environmental samples including surface swabs, air or water isolates.

The simplified method of sample preparation provided herein is used in a context wherein the RNA prepared from a single surface or environmental sample (swab, air or water) is used to identify the presence of 10-50 different viral pathogens in parallel. The highly parallel nucleic acid testing aspect of the present invention constitutes a major expansion of the information content of such testing. Measurements are performed on a single sample, such as obtained from a single surface swab, or a single air sample or a single water sample. Thus, the resulting preparation remains intact and sufficiently concentrated that it can support high throughput analysis of a large set (>>10) of candidate viral pathogens in parallel.

In one non-limiting example of the present invention, a diseased plant sample manifesting, for example, some sort of “wilt” may be tested simultaneously for all plant viruses that might reasonably produce such symptoms from a single plant isolate, thereby establishing which of many possible viruses is causing the observed plant symptoms. Thus, the methods provided herein provide in less than two hours a PCR amplified derivative of the original cDNA, which may in turn be analyzed by microarray testing in less than 4 additional hours to yield time sensitive data as to the cause of an infection from among 20 candidate plant pathogens, which may used to treat a rapidly-expanding crop infection.

In another non-limiting example, a human patient sample, such as a nasal swab from an individual presenting with symptoms of respiratory viral infection, as might have arisen from Influenza A or Influenza B or SARS or COVID-19 or some other coronavirus, may be utilized. The swab may be tested for all respiratory viruses simultaneously which might reasonably produce such symptoms from a single swab, thereby establishing which of many possible viruses is actually causing the observed patient symptoms. Thus, the present invention provides in less than six hours, detailed, time sensitive data as to the cause of a human respiratory infection, from among, but not limited to, 6 candidate human respiratory pathogens. This is useful to track the spread of infection, as in epidemiology, or to determine whether pharmaceutical treatment, intensive care or isolation is warranted, as in clinical virology.

In yet another non-limiting example, an air sample, for example, at least one collected in a hospital to test for the presence of aerosolized viruses such as Influenza A, Influenza B, SARS, or COVID-19 or some other Coronavirus may be utilized. The viral content of the air is collected on an air filter or by direct transfer to a collection fluid, which is tested for all respiratory viruses that might be in the air, from a single air sample, thereby establishing which of many possible viruses is actually present in the air.

Thus, the present invention provides in less than six hours detailed, time sensitive data as to the cause of viral infections in plants, viral respiratory infections in humans or the distribution of viruses in the air, all useful to track the spread of infection. Using prior art methods of viral RNA analysis, it would take 72-96 hours to generate such data. The present invention enables such analysis in less than six hours (about 1/10^{th} the time) and at about 1/10th the cost.

The following examples are given for the purpose of illustrating various embodiments of the invention and are not meant to limit the present invention in any fashion.

### EXAMPLE 1

### Isolation of a Crude Nucleic Acid Preparation from RNA Viruses

The present invention is based on the physical chemistry of such RNA viruses.

### Viruses Form Stable Sols

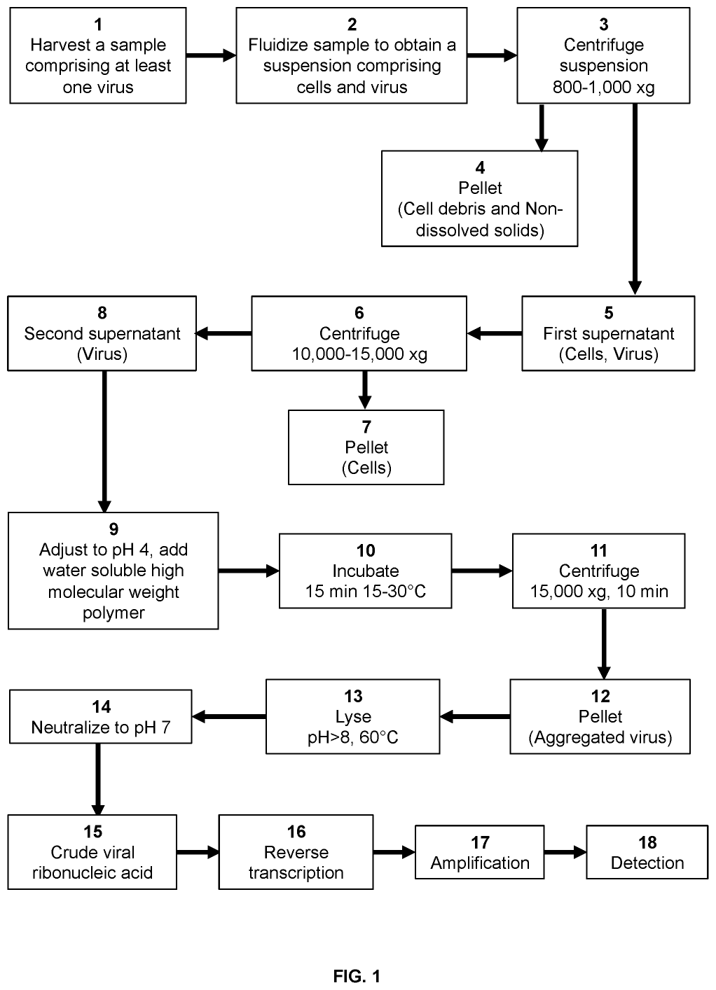

As a class, the RNA viruses form stable suspensions (sols) in aqueous solutions near physiological pH (pH 7) and ionic strength (150 mM-170 mM). Suspension stability is based on their relatively small size (<1 μm) and their negative surface charge attributed to coat proteins on their surface, which confer a negative zeta potential to most viral particles. As described in FIG. 1, at around pH 4 to pH 5, the surface charge of the coat proteins in RNA viruses neutralize, because they possess in general an isoelectric pH between pH 4 and pH 5. When the viral surface charge density becomes close to zero, the sol becomes unstable which results in aggregation of virus particles due to adsorption of virus particles to each other in the absence of the (ordinary) repulsive negative charge that is seen near neutral pH.

Near neutral pH, even though virus particles maintain a negative surface charge, the magnitude of the zeta potential (i.e. effective surface charge) can be reduced by addition of screening counterions like NaCl, KCl and sodium phosphate or sodium acetate among others, which reduce the Debye layer on the viral particle surface. Under those conditions the RNA viruses aggregate as a result of such counterion screening.

### Viruses Form Stable Sols in Aqueous Solution, but not when Concentrated or Suspended in Presence of High Molecular Weight Polymers

Even at pH 7 and an ionic strength of about 150 mM to about 200 mM, which maintains an adequate surface charge on their coat proteins, suspended viral sols can be induced to aggregate if the mass density of the virus is raised to a point which exceeds the dissociation constant for virus-virus physical association (typically in the 1mg/ml range). Below that critical virus concentration, aggregation may be induced by addition of (non-viral) particulates with a similar size (50 nm-500 nm) and surface pKa (around pH 4) to increase the total concentration of particles to be aggregated, thereby driving the viral aggregation process.

Below that critical virus concentration, aggregation may be additionally induced by addition of a high mass density (typically 1%-10%) of a water soluble high molecular weight polymer like a polyethylene glycol (e.g. PEG 8000) or dextran or dextran sulfate. As described in FIG. 1, addition of such a water soluble polymer creates an aqueous polymer solution with two phases in direct proximity—(i) the included volume phase, which is that fraction of the water phase which resides within the radius of gyration of each polymer molecule; and (ii) the excluded volume phase, which is that fraction of the water phase which lies outside the radius of gyration of each polymer molecule, where the viral particles must reside.

Because viruses are much larger than the radius of gyration of such water-soluble polymers, they are expelled (partitioned) into the excluded volume. At a high mass density of such polymers (e.g. in the range of 1%-10% for PEG 8000) individual viral particles reside in the smaller effective volume of the excluded volume fraction, and thus in a thermodynamic sense, have become concentrated, causing aggregation. At most achievable viral concentrations, the partitioning equilibrium into the excluded volume and the subsequent aggregation of viral particles remains inefficient at room temperature. For this reason, the use of water-soluble polymers in the prior art is almost exclusively combined with cooling between 4° C. and −20° C., to facilitate viral aggregation. The present invention obviates that need for refrigerated cooling.

### Individual Virus Particles Sediment Slowly, but Much More Rapidly upon Aggregation

Because RNA viruses have a dimension <1 μm and comprise protein and RNA, which defines a density only slightly greater than water (i.e. n>1) they can be made to sediment only at high speeds, typically >50,000×g, which is not attainable on a standard bench top centrifuge. Aggregation of the virus particles on the other hand allows their sedimentation in a standard laboratory centrifuge at a speed of about 15,000×g.

It is very well known in the art that when a high molecular weight polymer like PEG8000 is added to an RNA virus preparation, then cooled to 4° C. or −20° C., viral aggregation will occur, allowing them to be harvested as a pellet by low speed centrifugation. It is also well known that if the pH of a solution is adjusted between pH 4 and pH 5, followed by cooling to 4° C. to −20° C., viral aggregation occurs allowing them to be harvested as a pellet by low speed centrifugation.

A major limitation imposed in either prior art scenario is the requirement that the viral sample be incubated for a long period of time at 4° C. or −20° C. in order to allow sufficient low temperature aggregation to support low speed centrifugation.

Thus, a key attribute of the present invention is the discovery of a novel combination of the various physical properties of RNA viruses (i.e., viral surface neutralization near pH 4, polymer exclusion to achieve local concentration increase and in some instances the addition of non-viral particles) which in the aggregate enable rapid isolation of RNA viruses from plant, animal or environmental samples (such as swabs, water, air) by low speed centrifugation at room temperature. This is achieved by the use of a first centrifugation step designed to remove the preponderance of contaminating plant, animal, bacterial and fungal matter prior to the induction of viral aggregation. See FIG. 1.

The present invention also takes advantage of the fact that, once an enriched viral pellet is obtained via room temperature aggregation and centrifugation, the resulting viral pellet can be lysed by heating at an elevated pH, followed by direct enzymatic conversion of the RNA in the crude viral lysate to cDNA using reverse transcriptase (RT) to generate a cDNA without the need for further purification. The reverse transcription (cDNA) product is then combined with a first “enrichment” PCR reaction, to greatly increase the abundance of the cDNA product to an extent such that the PCR-amplified cDNA may be then used for any nucleic acid testing including PCR, qPCR, Next Generation Sequencing (NGS) or a preferred implementation comprising a Labelling PCR reaction, followed by microarray hybridization.

Thus, the present invention enables an RNA virus to be isolated then analyzed from an infected plant sample or animal or human sample or an environmental sample (swabs, air, water) without refrigeration or RNA purification in a form suitable for many types of nucleic acid analysis, including qPCR or PCR-CE or highly parallel analysis such as Next Generation Sequencing or microarray analysis.

### EXAMPLE 2

### Reagents

1. Cannabis or Hemp (infected plant matter).

2. Tobacco Mosaic Virus and Cucumber Mosaic Virus (viral agent).

### General Procedure

Step 1. The virus is released from the infected plant by mechanical disruption in physiological saline (PBS).

Step 2. Contaminating plant matter, bacterial pathogens and fungal pathogens are removed from the PBS isolate by a first centrifugation at 5,000×g.

Step 3. The pH of the supernatant from the previous step, is adjusted to pH 4.2 with Na Acetate, PEG 8000 was added to 4% (w/v), to induce viral aggregation within 15 min of incubation at room temperature (15° C.-30° C.).

Step 4. The virus aggregate is sedimented by a second centrifugation at 15,000×g for 10 min.

Step 5. The sedimented pellet is lysed in 4-(Cyclohexyl amino)-1-butanesulfonic acid/Ethylenediamine tetra acetic acid (CABS-EDTA) buffer-pH 10 containing 0.1% Tween-20 by heating to 60° C. to lyse the virion and release the RNA for analysis.

Step 6. One pot RT-PCR of the viral lysate, such as with Invitrogen SuperScript IV One-Step RT-PCR System is used to amplify viral RNA regions of interest to yield an abundance of cDNA based PCR amplicons for subsequent DNA-based analysis such as qPCR, sequencing, or DNA microarray hybridization analysis or other methods of hybridization analysis such as Luminex Beads.

### EXAMPLE 3

### Room Temperature Isolation of Plant Virus by Centrifugation and RT-PCR

One gram of plant matter infected or doped with a pure virus stock (1, FIG. 1) is placed in a stomacher bag with filter. PBS (10 ml) was added. The plant was crushed and hydrated by hand to make a crude fluid homogenate (2, FIG. 1). The fluid phase was decanted from the stomacher bag through its 100 μm filter into a 15 ml conical storage tube. 10 ml of the filtrate was centrifuged at 1,000×g (3, FIG. 1) in a 15 ml microfuge tube to remove cell debris and solids (4, FIG. 1) . The supernatant (5, FIG. 1) was pipetted into a new 15 ml tube and centrifuged at 15,000×g (6, FIG. 1) for 5 min to remove intact cells (7, FIG. 1).

The supernatant (8, FIG. 1) is decanted and mixed with 1 volume of 8% PEG 8000 to obtain a final concentration of 4% PEG 8000 (a water soluble high molecular weight polymer) and pH adjusted to 4 by addition of sodium acetate (9, FIG. 1). The mixture is incubated for 15 min at room temperature (10, FIG. 1), following which, it is centrifuged at 15,000×g for 10 min (11, FIG. 1) obtain a pellet (12, FIG. 1) containing the aggregated virus.

The viral pellet is resuspended in 10 mM CABS (pH 10) 1 mM EDTA containing 0.1% Tween 20 and heated to 60° C. for 10 min to lyse the virus (13, FIG. 1). The pH of lysed virus is neutralized to 7 (14, FIG. 1) to obtain a crude viral ribonucleic acid (15, FIG. 1). 2 μL of the lysate is mixed with 50 μL of a modified Superscript One-Step RT-PCR reaction buffer and a RT-PCR reaction is performed (16, FIG. 1) using reverse transcriptase and primer pairs specific for the RNA virus of interest (Table 1). The complementary DNA generated is amplified (17, FIG. 1) and the virus detected (18, FIG. 1). In a preferred implementation, microarray hybridization analysis, 1 μL of amplicons obtained from the previous step is used in a Labeling PCR reaction using fluorescent labeled primer pairs (Table 2) to obtain fluorescent labeled amplicons. The amplicons thus obtained are hybridized on a DNA Microarray to which are attached virus sequence specific nucleic acid probes.

TABLE 1
Reverse transcription primers

| | | | Reference, |
|---|---|---|---|
| | | | Collection |
| SEQ ID NO. | Target | Primer Sequence | method |
| SEQ ID NO: 1 | COVID-19 N1 domain | GACCCCAAAATCAGCGAAAT | 2, |
| | Forward primer | | Nares |
| | | | swab |
| SEQ ID NO: 2 | COVID-19 N1 domain | TCTGGTTACTGCCAGTTGAAT | 2, |
| | Reverse primer | CTG | Nares |
| | | | swab |
| SEQ ID NO: 3 | COVID-19 N2 domain | TTACAAACATTGGCCGCAAA | 2 |
| | Forward primer | | Nares |
| | | | swab |
| SEQ ID NO: 4 | COVID-19 N2 domain | GCGCGACATTCCGAAGAA | 2, |
| | Reverse primer | | Nares |
| | | | swab |
| SEQ ID NO: 5 | COVID-19 N3 domain | GGGAGCCTTGAATACACCAA | 2, |
| | Forward primer | AA | Nares |
| | | | swab |
| SEQ ID NO: 6 | COVID-19 N3 domain | TGTAGCACGATTGCAGCATT | 2, |
| | Reverse primer | G | Nares |
| | | | swab |
| SEQ ID NO: 7 | Influenza A CDC | GACCRATCCTGTCACCTCTG | 3-5, |
| | Forward primer | AC | Nares |
| | | | swab |
| SEQ ID NO: 8 | Influenza A CDC | AGGGCATTYTGGACAAAKCG | 3-5, |
| | Reverse primer | TCTA | Nares |
| | | | swab |
| SEQ ID NO: 9 | Influenza B CDC | TCCTCAACTCACTCTTCGAGC | 3, |
| | Forward primer | G | Nares |
| | | | swab |
| SEQ ID NO: 10 | Influenza B CDC | CGGTGCTCTTGACCAAATTG | 3, |
| | Reverse primer | G | Nares |
| | | | swab |
| SEQ ID NO: 11 | Tobacco mosaic virus | ATTAGACCCGCTAGTCACAG | 1, |
| | Forward primer | CAC | Plant Wash |
| SEQ ID NO: 12 | Tobacco mosaic virus | GTGGGGTTCGCCTGATTTT | 1, |
| | Reverse primer | | Plant Wash |

TABLE 2
PCR primers

| | | | Reference, |
|---|---|---|---|
| | | | Collection |
| SEQ ID NO. | Target | Primer Sequence | method |
| SEQ ID NO: 13 | COVID-19 N1 domain | GACCCCAAAATCAGCGA | 2, |
| | Forward primer | AAT | Nares |
| | | | swab |
| SEQ ID NO: 14 | COVID-19 N1 domain | TCTGGTTACTGCCAGTTG | 2, |
| | Reverse primer | AATCTG | Nares |
| | | | swab |
| SEQ ID NO: 15 | COVID-19 N2 domain | TTACAAACATTGGCCGCA | 2, |
| | Forward primer | AA | Nares |
| | | | swab |
| SEQ ID NO: 16 | COVID-19 N2 domain | GCGCGACATTCCGAAGA | 2, |
| | Reverse primer | A | Nares |
| | | | swab |
| SEQ ID NO: 17 | COVID-19 N3 domain | GGGAGCCTTGAATACAC | 2, |
| | Forward primer | CAAAA | Nares |
| | | | swab |
| SEQ ID NO: 18 | COVID-19 N3 domain | TGTAGCACGATTGCAGC | 2, |
| | Reverse primer | ATTG | Nares |
| | | | swab |
| SEQ ID NO: 19 | Influenza A CDC | GACCRATCCTGTCACCT | 3-5, |
| | Forward primer | CTGAC | Nares |
| | | | swab |
| SEQ ID NO: 20 | Influenza A CDC | AGGGCATTYTGGACAAA | 3-5, |
| | Reverse primer | KCGTCTA | Nares |
| | | | swab |
| SEQ ID NO: 21 | Influenza B CDC | TCCTCAACTCACTCTTCG | 3, |
| | Forward primer | AGCG | Nares |
| | | | swab |
| SEQ ID NO: 22 | Influenza B CDC | CGGTGCTCTTGACCAAA | 3, |
| | Reverse primer | TTGG | Nares |
| | | | swab |
| SEQ ID NO: 23 | Tobacco mosaic virus | ATTAGACCCGCTAGTCA | 1, |
| | Forward primer | CAGCAC | Plant Wash |
| SEQ ID NO: 24 | Tobacco mosaic virus | GTGGGGTTCGCCTGATT | 1, |
| | Reverse primer | TT | Plant Wash |

### EXAMPLE 4

### PRV-DetectX Versus q-RT-PCR

Test Content: PathogenDx has completed design and begun manufacture of a microarray-based Pan Respiratory Virus test (“PRV-DetectX”) to be submitted for FDA EUA review, with SARS-CoV-2 as the analyte plus multiple coronavirus targets including, SARS-CoV, MERS-CoV, CoV 229E, CoV OC43, CoV NL63, CoV HKU1, and influenza virus A and influenza virus B as internal specificity controls (Table 3). The same microarray assay also includes 2 positive controls—RNAse P and a Synthetic Quantitative Standard.

The information content of PRV-DetectX is about 10-fold greater than that of the current q-RT-PCR tests. The extra content available in the microarray format allows a very large panel of COVID-19 target sites to be measured in parallel and in triplicate. Although the other six coronavirus targets and the two influenza targets also included in PRV-DetectX are being used as controls in the present COVID-19 testing, PRV-DetectX is as a universal screening tool for both coronavirus and influenza.

TABLE 3
PRV-DetectX Viral Targets, PCR Primer
and Microarray Probe Complexity

| | | Microarray | | |
|---|---|---|---|---|
| Viral Target | Target Sites/Virus | Probes | PCR Primers | |
| SARS-CoV-2 | N1, N2, N3, | 24 | 6 | sets |
| COVID-19 | ORF1ab, RdRp, E | | | |
| SARS-CoV | N, 1ab | 4 | | |
| MERS-CoV, | N, 1ab | 2 | 2 | sets |
| CoV 229E, | N, 1ab | 2 | 2 | sets |
| CoV OC43, | N, 1ab | 2 | 2 | sets |
| CoV NL63, and | N, 1ab | 2 | 2 | sets |
| CoV HKU1) | N, 1ab | 2 | 2 | sets |
| Influenza virus | M, NS1 | 2 | 2 | sets |
| A-type | | | | |
| Influenza virus | M, NS1 | 2 | 2 | sets |
| B-type | | | | |
| Positive (RNA) | RNase P | 2 | 1 | set |
| Extraction Control | | | | |
| SARS-CoV-2 (DNA) | 1 Synthetic | | | |
| Positive Control | | | | |

| | | | |
|---|---|---|---|
| 10 Targets | 19 Targets | 48 Probes | 20 PCR |
| | | | Primer Sets |

Throughput: PRV-DetectX is optimized to provide all its viral target data from a single nares or throat swab. It of course can be noted that the information content of PRV-DetectX could be emulated by q-RT-PCR. However, inspection of the last column of Table 3 reveals that it would take about 20 such q-RT-PCR reactions (about 20 primer-probe triplets) to match the information content of PRV-DetectX which only takes 1 kit. Twenty such triplets would almost certainly require more RNA than can be obtained from a single swab.

Specificity: For each of the 6 unique SARS-CoV-2 target loci in PRV-DetectX [N1, N2, N3, ORF1ab, RdRp, E] there is one microarray probe for each locus (12 specific probes in total) and homologous probes with 20% of intentional base mismatching (i.e. there are 12 mismatched specificity probes). In that format, a positive COVID-19 signal for any one of the set of six loci, is only valid if it possesses a fluorescence signal strength of >10× background (>10,000 RFU) while at the same time and in the same microarray, the mismatched specificity probe generates a signal less than 2× background (<2,000 RFU). That is, “real” PRV-DetectX signals generate “strong” hybridization signals (>10,000 RFU) under conditions where the matched specificity control generates a “weak” hybridization signal that is at least 10× smaller. Such direct, explicit COVID-19 analyte specificity is unique to PRV-DetectX and cannot easily be obtained during ordinary q-RT-PCR.

Sensitivity: The limit of detection (LoD) for PRV-DetectX is less than 1 viral genome per assay and as such is more than 10× more sensitive than q-RT-PCR. This is due to the unique, patented properties of the tandem PCR and microarray technologies upon which PRV-DetectX is based. Such a >10× sensitivity enhancement enables (for the first time) the ability to detect and speciate COVID-19 at less than 100 virus particles per swab, which according to the literature, is roughly 10× greater sensitivity than any known q-RT-PCR reaction. The current standard for COVID-19 analysis in terms of LoD and species resolution can be improved by at least an order of magnitude in the PRV-DetectX test relative to that obtained by any known q-RT-PCR test.

The following references are cited herein.

1. Yang et al. Sensors (Basel). 12:16685-16694, 2012.

2. Centers for Disease Control and Prevention (CDC) Atlanta, Ga. Novel Coronavirus (2019-nCoV) Real-time rRT-PCR Panel Primers and Probes 2019.

3. Selvaraju S B and Selvarangan R. J Clin Microbiol. 48:3870-3875, 2010.

4. Scoizec et al. Front. Vet. Sci. 5:15, 2018.

5. Spackman et al. J Clin Microbiol. 40:3256-3260, 2002.

What is claimed is:

1. A method for isolating at least one virus from a sample, comprising the steps of:
obtaining the sample;
fluidizing the sample to produce a suspension comprising the virus;
centrifuging the suspension to obtain a first supernatant comprising the virus;
centrifuging the first supernatant to obtain a second supernatant comprising the virus;
incubating the second supernatant with a water soluble high molecular weight polymer or non-viral particles or a combination thereof;
adjusting the pH to less than pH 6 to form an aggregated virus;
centrifuging the aggregated virus to obtain a pellet comprising the aggregated virus;
adding a lysis buffer to the pellet;
heating the pellet in the lysis buffer; and
neutralizing the pH to 7 to produce a crude neutralized lysate comprising the one virus.

2. The method of claim 1, further comprising analyzing the crude neutralized lysate to detect the presence of the virus in the sample, the method comprising the steps of:
isolating, from the crude neutralized lysate, viral nucleic acids;
performing a reverse transcription reaction using the isolated viral nucleic acids as a template to obtain virus-specific complementary deoxyribonucleic acids (cDNA);
amplifying, in at least one amplification, a target nucleotide sequence in the virus-specific cDNA using at least one primer pair selective for the virus to generate a plurality of virus-specific amplicons; and
detecting the presence of the at least one virus in the sample via hybridization of the plurality of virus-specific amplicons to a plurality of nucleic acid probes each specific for the virus.

3. The method of claim 2, wherein each of the primer pairs further comprises a first fluorescent label, said method generating a plurality of first fluorescent labeled virus-specific amplicons.

4. The method of claim 3, wherein the amplifying step and the detecting step comprise:
performing one of a first polymerase chain reaction (PCR) amplification followed by a second PCR amplification, an isothermal amplification or the reverse transcription reaction and the first polymerase chain reaction amplification together, each of said amplifications using the at least one virus-specific complementary deoxyribonucleic acid as template and at least one first fluorescent labeled primer pair selective for the virus to generate the first fluorescent labeled virus-specific amplicons;
hybridizing the first fluorescent labeled virus specific amplicons to a microarray comprising a plurality of nucleic acid probes each having a sequence corresponding to sequence determinants in a plurality of virus-specific ribonucleic acids;
washing the microarray at least once; and
imaging the microarray to detect a first fluorescent signal image corresponding to the first fluorescent labeled virus-specific amplicons.

5. The method of claim 4, wherein the amplifying step comprises:
performing the first polymerase chain reaction using the at least one virus-specific complementary deoxyribonucleic acid as template and at least one unlabeled primer pair selective for the virus to generate virus-specific amplicons corresponding to the viral ribonucleic acid; and
performing the second polymerase chain reaction using the virus-specific amplicons as template and the at least one first fluorescent labeled primer pair having a sequence complementary to a region in the virus-specific amplicons to generate the first fluorescent labeled virus specific amplicons.

6. The method of claim 4, wherein the microarray comprises a plurality of bifunctional polymer linkers each covalently attached to the microarray and to one of the nucleic acid probes and each comprising a second fluorescent label covalently attached thereto, the method further comprising:
detecting in the imaging step, a second fluorescent signal image corresponding to the second fluorescent labeled bifunctional polymer linkers;
superimposing the first fluorescent signal image with the second fluorescent signal image to obtain a superimposed signal image; and
comparing the sequence of the nucleic acid probe at one or more superimposed signal positions on the microarray with a database of signature sequence determinants for a plurality of viral ribonucleic acid thereby identifying the virus in the sample.

7. The method of claim 6, wherein the second fluorescent label in the fluorescent labeled bifunctional polymer linker is different from the first fluorescent label in the fluorescent labeled primer pair.

8. The method of claim 1, wherein the fluidizing step comprises mechanically disrupting the sample in a disruption buffer.

9. The method of claim 1, wherein the harvesting step comprises washing the sample or dispersing a swab in a liquid.

10. The method of claim 1, wherein the water soluble high molecular weight polymer is at a concentration of 1% to 10% w/v.

11. The method of claim 1, wherein the water soluble high molecular weight polymer is selected from the group consisting of polyethylene glycol, dextran and dextran sulfate.

12. The method of claim 1, wherein the non-viral particle is a ceramic particle.

13. The method of claim 12, wherein the non-viral particle comprises SiO₂.

14. The method of claim 1, wherein the non-viral particle has a dimension from about 50 nm to about 500 nm.

15. The method of claim 1, wherein the lysis buffer has a pKa from about 7 to about 10.

16. The method of claim 1, wherein the sample is an aerosol.

17. The method of claim 1, wherein the sample is a tissue or cells from a human, a plant, an animal, a bacteria, a fungus, an algae.

18. The method of claim 1, wherein the sample is a surface swab, skin swab, a nares swab, a milk sample, a blood sample, a sputum sample, a mucus sample, an urine sample or a feces sample.

19. The method of claim 1, wherein the virus is a single stranded RNA virus or a double stranded RNA virus.

20. The method of claim 1, wherein the virus is selected from the group consisting of a human immunodeficiency virus, an influenza virus, a coronavirus, a COVID-19, a hepatitis C virus, a dengue virus, a norovirus, a rotavirus, a measles virus, a tobacco mosaic virus, a tobacco ringspot virus, a hemp mosaic virus, a cucumber mosaic virus, a potyvirus, a beet curly top virus, a tobacco streak virus, an alfalfa mosaic virus, an arabis mosaic virus, a cannabis cryptic virus, a hop latent virus, a hemp streak virus and a cauliflower mosaic virus.


SEQUENCE LISTING (24 sequences)

SEQ ID NO: 1 - DNA, 20 bp, Artificial sequence
  Forward primer for COVID-19 N1 domain
gaccccaaaatcagcgaaat

SEQ ID NO: 2 - DNA, 24 bp, Artificial sequence
  Reverse primer for COVID-19 N1 domain
tctggttactgccagttgaatctg

SEQ ID NO: 3 - DNA, 20 bp, Artificial sequence
  Forward primer for COVID-19 N2 domain
ttacaaacattggccgcaaa

SEQ ID NO: 4 - DNA, 18 bp, Artificial sequence
  Reverse primer for COVID-19 N2 domain
gcgcgacattccgaagaa

SEQ ID NO: 5 - DNA, 22 bp, Artificial sequence
  Forward primer for COVID-19 N3 domain
gggagccttgaatacaccaaaa

SEQ ID NO: 6 - DNA, 21 bp, Artificial sequence
  Reverse primer for COVID-19 N3 domain
tgtagcacgattgcagcattg

SEQ ID NO: 7 - DNA, 22 bp, Artificial sequence
  Forward primer for influenza A
gaccratcctgtcacctctgac

SEQ ID NO: 8 - DNA, 24 bp, Artificial sequence
  Reverse primer for influenza A
agggcattytggacaaakcgtcta

SEQ ID NO: 9 - DNA, 22 bp, Artificial sequence
  Forward primer for influenza B
tcctcaactcactcttcgagcg

SEQ ID NO: 10 - DNA, 21 bp, Artificial sequence
  Reverse primer for influenza B
cggtgctcttgaccaaattgg

SEQ ID NO: 11 - DNA, 23 bp, Artificial sequence
  Forward primer for tobacco mosaic virus
attagacccgctagtcacagcac

SEQ ID NO: 12 - DNA, 19 bp, Artificial sequence
  Reverse primer for tobacco mosaic virus
gtggggttcgcctgatttt

SEQ ID NO: 13 - DNA, 20 bp, Artificial sequence
  Forward primer for COVID-19 N1 domain
gaccccaaaatcagcgaaat

SEQ ID NO: 14 - DNA, 24 bp, Artificial sequence
  Reverse primer for COVID-19 N1 domain
tctggttactgccagttgaatctg

SEQ ID NO: 15 - DNA, 20 bp, Artificial sequence
  Forward primer for COVID-19 N2 domain
ttacaaacattggccgcaaa

SEQ ID NO: 16 - DNA, 18 bp, Artificial sequence
  Reverse primer for COVID-19 N2 domain
gcgcgacattccgaagaa

SEQ ID NO: 17 - DNA, 22 bp, Artificial sequence
  Forward primer for COVID-19 N3 domain
gggagccttgaatacaccaaaa

SEQ ID NO: 18 - DNA, 21 bp, Artificial sequence
  Reverse primer for COVID-19 N3 domain
tgtagcacgattgcagcattg

SEQ ID NO: 19 - DNA, 22 bp, Artificial sequence
  Forward primer for influenza A
gaccratcctgtcacctctgac

SEQ ID NO: 20 - DNA, 24 bp, Artificial sequence
  Reverse primer for influenza A
agggcattytggacaaakcgtcta

SEQ ID NO: 21 - DNA, 22 bp, Artificial sequence
  Forward primer for influenza B
tcctcaactcactcttcgagcg

SEQ ID NO: 22 - DNA, 21 bp, Artificial sequence
  Reverse primer for influenza B
cggtgctcttgaccaaattgg

SEQ ID NO: 23 - DNA, 23 bp, Artificial sequence
  Forward primer for tobacco mosaic virus
attagacccgctagtcacagcac

SEQ ID NO: 24 - DNA, 19 bp, Artificial sequence
  Reverse primer for tobacco mosaic virus
gtggggttcgcctgatttt